(19) United States
(12) Patent Application Publication
(10) Pub. No.: US 2019/0046042 A1
(43) Pub. Date: Feb. 14, 2019

(54) PHOTOACOUSTIC PROBE

(71) Applicant: CANON KABUSHIKI KAISHA, Tokyo (JP)

(72) Inventor: TOSHINOBU TOKITA, Yokohama-shi (JP)

(21) Appl. No.: 16/055,590

(22) Filed: Aug. 6, 2018

(30) Foreign Application Priority Data
Aug. 10, 2017 (JP) 2017-155556

(51) Int. Cl. A61B 5/00; A61B 8/00; G01N 21/17; G01N 29/24

(52) U.S. Cl. CPC A61B 5/0095; A61B 8/4444; G01N 29/2418; A61B 8/4281; G01N 21/1702; A61B 5/0035

(57) ABSTRACT

A photoacoustic probe is used, which includes: a reception element configured to receive an acoustic wave generated in and propagated from an object which is irradiated with light; an elastic body configured to contact with the object when receiving the acoustic wave and to diffuse or transmit the light having a wavelength that generates the acoustic wave; an acoustic matching layer disposed between the elastic body and the reception element; and a reflection film disposed between the acoustic matching layer and the elastic body.

### BACKGROUND OF THE INVENTION

### Field of the Invention

The present invention relates to a photoacoustic probe.

### Description of the Related Art

Photoacoustic imaging (PAI) is receiving attention as a method of specifically imaging micro vessels which are generated due to cancer or inflammation. PAI is a system in which an illumination light (near infrared) is radiated to an object, and an acoustic wave (photoacoustic wave) generated from inside the object is received by a probe, and is imaged. The signal receiving portion of the probe normally converts the pressure of the acoustic wave into an electric signal using a reception element, such as a piezoelectric element.

The acoustic impedance of the piezoelectric element and that of the object are considerably different, hence an acoustic matching layer is disposed in the probe. In many cases, an acoustic lens is disposed in the probe to focus the acoustic wave. However, if a part of the illumination light to illuminate the object contacts the acoustic lens, a photoacoustic wave is generated from the acoustic lens. When the photoacoustic wave, generated from the acoustic lens propagates into the object, is reflected and scattered, and is then received again by the probe, noise is generated and interrupts the target photoacoustic wave from the object to be acquired. The generation of this noise is a cause of dropping the contrast of the object information, and decreases the signal-to-noise ratio (SNR).

To solve this problem, U.S. Patent Application Publication No. 2016/0296121 discloses that a reflection film made of metal film is disposed on and around the acoustic lens. Further, when gold is used for the metal film, a parylene film is coated on the acoustic lens which is made of RTV (Room Temperature Vulcanizing) rubber, then nickel and gold are coated thereon so that the acoustic lens is not damaged or cracked. Then a parylene film is coated again on the gold. In this way, according to U.S. Patent Application Publication No. 2016/0296121, the generation of the photoacoustic wave from the acoustic lens is reduced by reflecting the light radiated to the acoustic lens using the metal film.

Japanese Patent No. 5855994 discloses that a light diffusion acoustic member, which contains such oxide particles as titanium oxide, is used for the reception surface of the probe represented by the acoustic lens. In Japanese Patent No. 5855994, the generation of the photoacoustic wave from the acoustic lens is reduced by decreasing the light absorption of the acoustic lens.

However, according to U.S. Patent Application Publication No. 2016/0296121, the parylene film must be coated to protect the acoustic lens and the metal film, which may drop the acoustic wave transmittance.

A problem of Japanese Patent No. 5855994 will be described with reference to FIG. 6. According to the photoacoustic probe 500 disclosed in Japanese Patent No. 5855994, the light source (emitting end) 506 emits an illumination light 507. A part of the illumination light 507 is absorbed by a light absorber inside an object 505, and generates a photoacoustic wave. Another part of the illumination light 507, however, wraps around and contacts the acoustic lens 503 via the object 505 or gel (not illustrated) disposed as an acoustic matching agent between the object 505 and the photoacoustic probe 500. Then, because of the light diffusing member disposed on the acoustic lens 503, light enters into the acoustic matching layer 502 disposed between the acoustic lens 503 and the piezoelectric element 501, and the photoacoustic wave is generated from the acoustic matching layer 502. If this photoacoustic wave generated from the acoustic matching layer 502 propagates into the object, is reflected and scattered, and is then received again by the probe, noise may be generated.

With the foregoing in view, it is an object of the present invention to improve the SNR of the photoacoustic imaging.

### SUMMARY OF THE INVENTION

The present invention provides a photoacoustic probe, comprising:

a reception element configured to receive an acoustic wave generated in and propagated from an object which is irradiated with light;

an elastic body configured to contact with the object when receiving the acoustic wave and to diffuse or transmit the light having a wavelength that generates the acoustic wave;

an acoustic matching layer disposed between the elastic body and the reception element; and

a reflection film disposed between the acoustic matching layer and the elastic body.

The present invention also provides a photoacoustic probe comprising an ultrasonic probe for ultrasonic echo measurement, and an attachment to be combined with the ultrasonic probe, wherein

the ultrasonic probe includes:
a transmission/reception element configured to transmit an ultrasonic wave to an object and to receive the ultrasonic wave reflected by the object;
a first elastic body contacting the object when the ultrasonic wave is transmitted to the object; and
an acoustic matching layer disposed between the first elastic body and the transmission/reception element, and wherein

the attachment includes:
a second elastic body contacting the object when the ultrasonic probe and the attachment are combined, and diffusing or transmitting light having a wavelength that generates the acoustic wave; and
a reflection film disposed between the second elastic body and the first elastic body when the ultrasonic probe and the attachment are combined.

Further features of the present invention will become apparent from the following description of exemplary embodiments with reference to the attached drawings.

### BRIEF DESCRIPTION OF THE DRAWINGS

FIG. 1 is a diagram depicting a general configuration of a photoacoustic imaging apparatus;

FIGS. 2A and 2B show diagrams depicting a configuration of a probe according to an embodiment;

FIGS. 3A and 3B show diagrams depicting another configuration of a probe according to an embodiment;

FIG. 4 is a diagram depicting a configuration of an attachment according to Embodiment 2;

FIG. 5 is a diagram depicting a light irradiation position according to Embodiment 3; and

FIG. 6 is a diagram depicting a problem of a prior art.

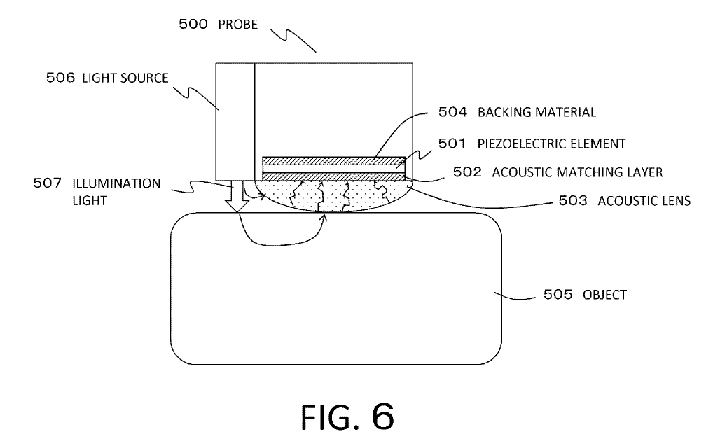

### DESCRIPTION OF THE EMBODIMENTS

Preferred embodiments of the present invention will be described with reference to the drawings. Dimensions, materials, shapes and relative positions of the components described below can be appropriately changed depending on the configuration and various conditions of the apparatus to which the invention is applied. Therefore, the following description is not intended to limit the scope of the present invention.

The present invention is related to a technique to detect an acoustic wave which propagates from an object, and generate and acquire characteristic information on the inside of the object (object information). Therefore, the present invention may be regarded as an object information processing apparatus or a control method thereof, or an acoustic wave apparatus or a control method thereof. Further, the present invention may be regarded as an object information processing method or a signal processing method. Furthermore, the present invention may be regarded as a program which causes an information processing apparatus including such hardware resources as a CPU and memory to execute these methods, or a computer readable non-transitory storage medium which stores the program.

The present invention is related more particularly to a probe used for photoacoustic imaging. Therefore, the present invention may be regarded as a probe, an ultrasonic probe, a photoacoustic probe or a light-induced ultrasonic probe or a manufacturing method thereof.

The present invention includes a photoacoustic apparatus utilizing a photoacoustic effect, which irradiates an object with light (electromagnetic wave), receives an acoustic wave generated inside the object, and acquires characteristic information on the object as image data. In this case, the characteristic information is information on characteristic values which are generated using signals originating from the received photoacoustic wave, and which correspond to a plurality of positions inside the object respectively.

The image data according to the present invention is a concept including various image data originating from the photoacoustic wave or a reflected wave. The image data indicates at least one characteristic information out of: the initial sound pressure of the photoacoustic wave, the absorption energy density, the absorption coefficient, the concentration of a substance constituting the object and the like. The image data indicating the concentration of a substance constituting the object is acquired based on the photoacoustic waves generated by radiating light beams having a plurality of mutually different wavelengths. The data indicating concentration includes an oxygen saturation degree, a value generated by weighting the oxygen saturation degree using an intensity value (e.g. absorption coefficient), a total hemoglobin concentration, an oxyhemoglobin concentration, and a deoxyhemoglobin concentration. The image data may indicate a glucose concentration, a collagen concentration, a melanin concentration, or the volume fraction of fat or water.

Based on the characteristic information at each position inside the object, a two-dimensional or three-dimensional characteristic information distribution is acquired. The distribution data can be generated as image data. In the present invention, three-dimensional volume data is generated by the image reconstruction as an example, but the present invention is not limited to this. The characteristic information may be determined as the distribution information at each position inside the object, instead of as numeric data. In other words, the initial sound pressure distribution, the energy absorption density distribution, the absorption coefficient distribution, the oxygen saturation degree distribution or the like may be determined.

The acoustic wave in the present invention is typically an ultrasonic wave, including an elastic wave called a “sound wave” and an “acoustic wave”. Such phases as ultrasonic wave or acoustic wave in this description, however, are not intended to limit the wavelengths of these elastic waves. A signal which is converted from an acoustic wave by a transducer or the like (e.g. electric signal) is called an “acoustic signal” or a “reception signal”. A signal converted from a photoacoustic wave is called a “photoacoustic signal”.

### Embodiment 1

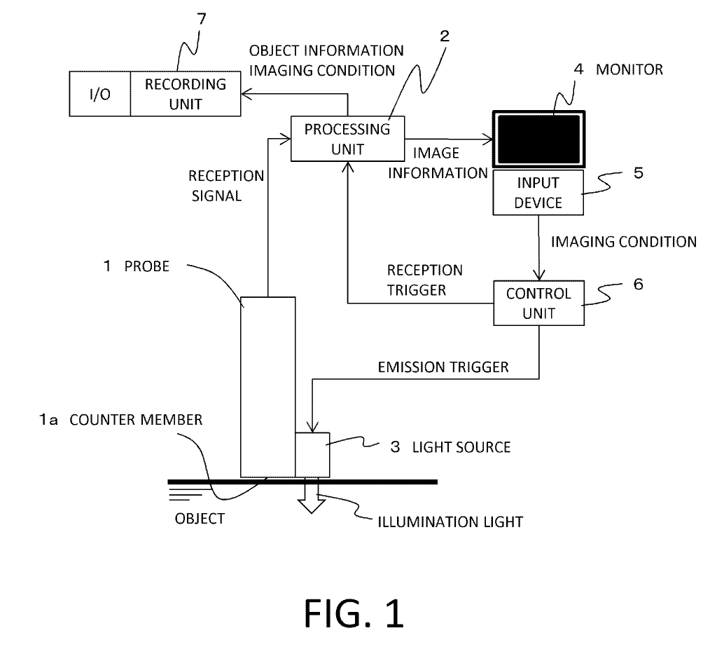

FIG. 1 is a schematic diagram depicting a general configuration of a photoacoustic imaging apparatus. The apparatus includes a probe 1, a processing unit 2, a light source 3, a monitor 4, an input device 5, a control unit 6 and a recording unit 7.

Apparatus Configuration

The probe 1 is a member that faces an object, and receives an acoustic wave at a counter member 1a, which is a reception surface, and converts the acoustic wave into an electric signal. The counter member 1a is a surface of an acoustic lens, for example. In the case when the photoacoustic wave is the reception target in PAI, the probe is also called a “photoacoustic probe”. The probe 1 may include a function to transmit the ultrasonic wave to the object, receive the ultrasonic wave reflected inside the object, and convert the ultrasonic wave into an ultrasonic signal (electric signal). The configuration of the probe 1 will be described in detail later.

The object may be a part of a living body, such as a breast and limb, or may be a phantom for calibration or an inorganic object. It is preferable to dispose an acoustic matching agent (e.g. gel, water) to enhance acoustic matching between the counter member 1a constituting the probe 1 and the object.

The processing unit 2 preforms such processing as amplification, A/D conversion, filtering and image reconstruction on the photoacoustic signal or ultrasonic signal received by the probe 1, so as to generate the characteristic information of the object. The processing unit 2 also performs beam forming when the probe 1 receives the photoacoustic wave, or receives/transmits the ultrasonic wave.

The processing unit 2 is normally constituted of components (e.g. CPU, GPU, A/D convertor, signal amplifier), circuits (e.g. FPGA, ASIC), and an information processing apparatus (e.g. PC, workstation) including these components and circuits. The processing unit may be constituted of one component or circuit, or may be configured by a combination of a plurality of components and circuits. The processing unit 2 includes a non-transitory recording medium, so as to store each processing performed by the object information acquiring method, as a program which the processing unit executes. The processing unit 2 uses an arbitrary image reconstruction method, such as phase regulating addition and a Fourier transform method.

The light source 3 radiates the illumination light to the object through an emitting end. When the light source 3 is combined with the probe 1, an LED or a semiconductor laser is preferable. Further, such a solid-state laser as Nd:YAG, Ti:sa, OPO and Alexandrite may be used for the light source. If a solid-state laser is used, it is preferable that the light is guided from a solid-state laser apparatus to the housing of the probe via an optical system, and the light is radiated from the emitting end (light emitting unit) disposed in the housing. Needless to say, the LED or the semiconductor laser may be disposed at a position that is distant from the probe 1, so that light is transmitted through an optical system.

The light source 3 is not limited to the above mentioned solid-state laser, semiconductor laser and LED, but a flash lamp may be used. For the optical system and the emitting end, any component, such as a lens, mirror, prism, optical filter and diffusion plate may be used, as long as light, emitted from the light source can be shaped in a desired shape and be radiated to the object. The light source 3 emits a pulsed light of several nsec to several hundred nsec to generate a photoacoustic signal. The pulsed light is preferably a rectangular pulse, but may be a Gaussian pulse. It is also preferable to use a wavelength-variable laser for the light source 3, or to combine a plurality of light sources 3 to enable to radiate light beams having a plurality of wavelengths. Thereby the oxygen saturation degree and the substance concentration inside the object can be calculated.

The monitor 4 displays the image information and the signal information generated by the processing unit 2. For the monitor 4, a liquid crystal display, an organic EL display or the like can be used. The input device 5 is used to set the imaging conditions to acquire the photoacoustic image and the ultrasonic image. The imaging conditions are, for example, a region of interest (ROI) setting, and the resolution of the image setting. For the input device 5, a pointing device (e.g. a mouse, trackball, touch panel), a keyboard and the like can be used. The monitor 4 and the input device 5 may be integrated with the photoacoustic imaging apparatus or may be provided separately.

The control unit 6 performs various controls based on the imaging conditions inputted by the input device 5. The imaging conditions are reflected in the processing unit 2. For example, when the photoacoustic image acquisition is started by the operation of the input device 5, the processing unit 2 stops the ultrasonic wave transmission, and emits the illumination light from the light source 3. At this time, the control unit 6 controls the emission trigger to perform the emission control of the light source 3, and the reception trigger to control the timing of the processing unit 2 receiving the signal and performing the processing. Further, when the ultrasonic image is acquired, the control unit 6] performs control using the input device 5, such as the selection of the imaging mode (e.g. B mode tomographic image, color doppler, power doppler), and the setting of the focus inside the object. According to this control, the processing unit 2 forms the beam of the ultrasonic wave, and transmits/receives the ultrasonic wave via the probe 1, so as to generate the image.

The control unit 6 is constituted of components (e.g. CPU, GPU), circuits (e.g. FPGA, ASIC) and an information processing apparatus, similarly to the processing unit 2. The control unit 6 and the processing unit 2 may be implemented by the same information processing apparatus.

The recording unit 7 records object information and various imaging conditions generated in the processing unit 2. The recording unit 7 may record reception signals before the image reconstruction. Further, the recording unit 7 can transfer, via I/O, object information and various imaging conditions to a computer in a medical facility connected by a network or external recording apparatus (not illustrated), such as a memory or a hard disk. For the recording unit 7, a non-volatile memory, and a storage circuit of the information processing apparatus constituting the processing unit 2 and the control unit 6, for example, may be used.

Configuration of Probe

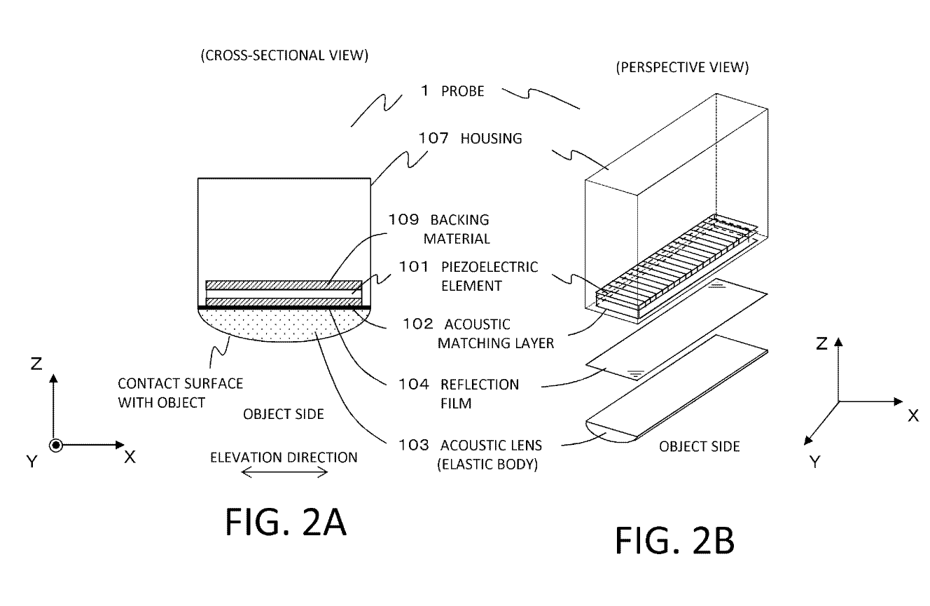

A configuration of the probe 1 will be described. FIG. 2A is a cross-sectional view when the probe 1 constituted of a linear array is viewed in the arrangement direction of the reception elements (piezoelectric elements 101). FIG. 2B is a schematic exploded perspective view depicting the probe 1. The probe 1 has a configuration in which each member is housed and installed in the housing 107. The probe 1 may be a hand held type which the user manually uses to scan, or a mechanical scanning type.

The probe 1 includes a plurality of piezoelectric elements 101, which are acoustic wave reception elements, disposed in a linear array. The piezoelectric element 101 converts the pressure of the ultrasonic wave into an electric signal. A backing material 109 is disposed near the piezoelectric elements 101 in order to suppress vibration. The acoustic impedance of the piezoelectric element 101 is 23 to 30 MRayls, and that of the object (living body) is about 1.5 MRayls. Since the acoustic impedances are completely different, an acoustic matching layer 102 is disposed so that reflection of the ultrasonic sound is reduced. The acoustic matching layer 102 may be a multilayer structure of at least two layers, but in FIGS. 2A and 2B, one layer of the acoustic matching layer 102 is illustrated for convenience. For the acoustic matching layer 102, resin, such as epoxy and polyethylene, can be used.

The surface of the acoustic lens 103 is convex, so as to focus the ultrasonic wave in the elevation direction of the probe 1. “Surface” here refers to the contact surface which faces and contacts the object. “Contact” here includes a case of contacting via the acoustic matching material or the like. The acoustic impedance of the acoustic lens 103 is preferably an intermediate between the acoustic impedance of the object and the acoustic impedance of the acoustic matching layer 102, and is preferably an elastic body made of resin material. For the resin material, rubber material, (e.g. silicon rubber, polyurethane and RTV) is particularly preferable. Such a plastic as polymethylpentene can also be used. In this embodiment, oxide particles constituted by titanium oxide or the like are dispersed in the material when the acoustic lens 103 is molded. The particle size is not more than 0.5 μm, and the content of the particles is not more than 0.5 wt %. It is particularly preferable that the content is in a range from 0.1 to 0.5 wt %. The elastic body diffuses or transmits light having a wavelength that generates the photoacoustic wave. “Transmit” here does not always mean that all lights are transmitted. The functions required for the elastic body can be implemented if at least a part of the lights is transmitted.

In this embodiment, a reflection film 104 is disposed at least between the acoustic matching layer 102 and the acoustic lens 103. For the reflection film 104, a metal film (e.g. gold) or a dielectric film is preferable. The reflectance of the reflection film 104 is preferably at least 90% with respect to the wavelength used for PAI. It is preferable that the reflection film 104 is directly formed on the object side surface of the acoustic matching layer 102. In the case of using a material of which direct forming is difficult, as in the case of gold, a film of primer may be formed first. The film may be formed on the acoustic lens 103 on the opposite surface of the surface that contacts with the object. Or a thin film may be adhered between the acoustic matching layer 102 and the acoustic lens 103.

According to one example, titanium oxide film is formed at a 100 Å thickness on a primer on the object side of the acoustic matching layer 102, and gold film is formed thereon at a 1500 Å thickness, whereby the reflection film 104 is generated. By this reflection film, at least 95% reflectance is acquired at about an 800 nm wavelength used for PAI. Then on the reflection film 104, an acoustic lens 103 made of silicon rubber, which contains titanium oxide particles (particle size: 0.21 μm) coated with aluminum oxide film at a 0.21 wt % concentration, is adhered.

When the acoustic wave is received, the piezoelectric element 101 sends the reception signal to the processing unit 2 via wires (not illustrated). For the piezoelectric element 101, a piezoelectric ceramic (e.g. BaTiO3, PZT, PbTiO3), a piezoelectric thin film (e.g. ZnO), a piezoelectric polymer film and the like can be used. Instead of the piezoelectric element, a capacitance conversion element cMUT or the like, which are commonly available, may be used.

Another Configuration Example of Probe

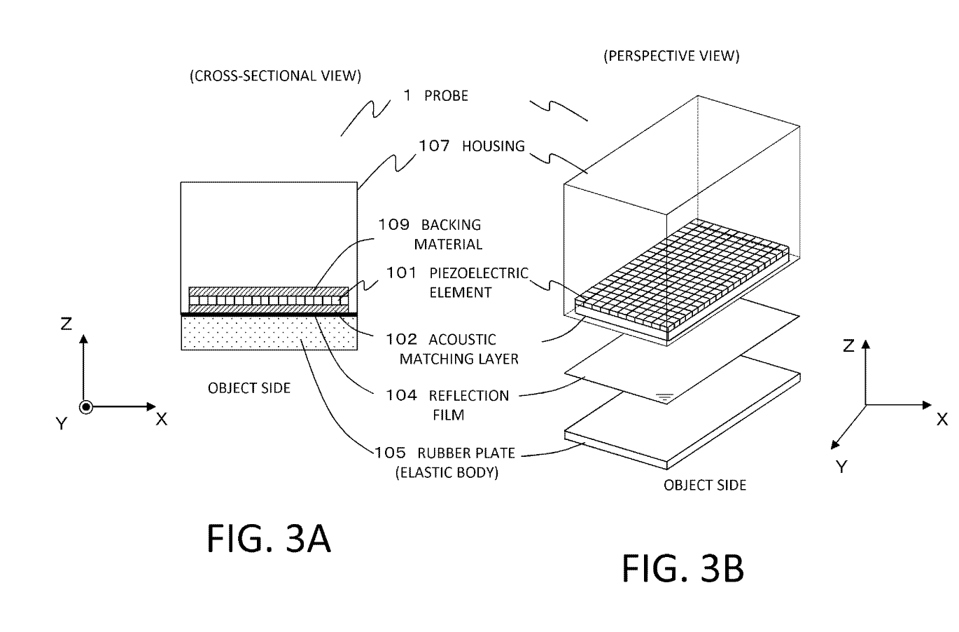

The above description is based on the assumption that the elastic body, which is a member facing the object, is the acoustic lens 103. However, the elastic body facing the object may not have the effect of the acoustic lens. FIGS. 3A and 3B show an example using a 2D array probe, where the piezoelectric elements 101 are arranged two-dimensionally, instead of the linear array. Description on a member that same as FIGS. 2A and 2B will be omitted.

In the case of FIG. 3A, a flat rubber plate 105 is used for the portion contacting with the object. This rubber plate 105 is made of material similar to the above mentioned acoustic lens 103. The acoustic lens 103 and the rubber plate 105 play the roles of compensating for the acoustic impedance difference. The thickness of the rubber plate 105 is preferably about ¼ that of the wavelength of the ultrasonic wave that propagates. Here the thickness is at least about ¼ that of the wavelength of the ultrasonic wave that propagates. According to one example, polyurethane (sound velocity: 1760 m/s) is used for the rubber plate 105, and the thickness of the rubber plate 105 is about 90 μm when the central frequency of the piezoelectric element 101 is 5 MHz. For the acoustic matching layer 102, epoxy resin (sound velocity: 2540 m/s, thickness: about 130 μm) is used.

The elastic body that faces the object is not limited to the acoustic lens 103 having a curvature, or to the plane rubber plate 105. The arrangement of the reception elements is not limited to a linear array or 2D array either, and may be a sector type, a convex type or a concave type. Furthermore, a single transducer may be effectively used as well. In this case, the present invention can also be applied to a photoacoustic microscopy (PAM) or to a photoacoustic signal acquiring apparatus.

In Embodiment 1 described above, even if the light emitted from the light source 3 is radiated to the elastic body that faces the object, the acoustic wave emitted from this elastic body decreases. Further, even if the illumination light enters into the elastic body, the light is reflected by the reflection film 104, hence the acoustic wave emitted from the acoustic matching layer 102 decreases. Since the acoustic waves emitted from the elastic body and the acoustic matching layer decrease respectively, the signals, which propagate into the object, are reflected and scattered, and are then received by the probe again, also decrease. As a result, the SNR of the photoacoustic signal improves, and the contrast of the object information acquired in PAI also improves. Furthermore, the reflection film 104 is between the acoustic matching layer 102 and the acoustic lens 103 or the rubber plate 105, and is not directly exposed to the outside, therefore the durability of the reflection film 104 against peeling, cracking and deterioration improves.

### Embodiment 2

In Embodiment 1 described above, oxide particles are contained in the elastic body, which is a member facing the object, and the reflection film 104 is disposed between the elastic body and the acoustic matching layer 102. This is a preferable configuration for the probe 1 used for PAI. In Embodiment 2, a case of using a probe 201 for ultrasonic echo measurement is used as the photoacoustic probe will be described.

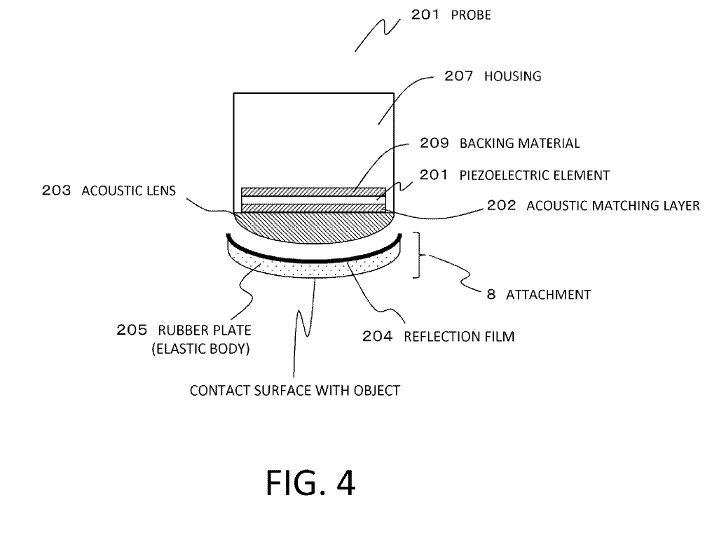

FIG. 4 is a diagram depicting a preferable method to perform PAI using the probe 201 for ultrasonic echo measurement. An acoustic lens 203 and an attachment 8, which are the major differences from FIGS. 2A and 2B, will primarily be described. The acoustic lens 203 in FIG. 4 is the probe 1 for ultrasonic echo measurement, and is not intended to radiate light, and the elastic body facing the object does not contain oxide particles. Therefore, if the probe 201 is directly used for PAI, a photoacoustic wave is generated from the acoustic lens 203.

When a standalone ultrasonic probe is used, the piezoelectric element 201 of Embodiment 2 functions as a transmission/reception element, which transmits the ultrasonic wave to the object and receives the ultrasonic echo generated by the ultrasonic wave that is reflected inside or on the surface of the object and then propagated. When the attachment and the ultrasonic probe are used in combination, the piezoelectric element 201 functions as a reception element which receives the photoacoustic wave.

In Embodiment 2, an attachment 8 is used for the probe 201. The attachment 8 is a rubber plate 205 on which the reflection film 104 is disposed. For reflection film 204, the reflection film described in FIGS. 2A and 2B is used, and is disposed at least on the surface of the rubber plate 205 opposite the surface that contacts the object. The rubber plate 205 is an elastic body in which oxide particles, similar to those of the acoustic lens described in FIGS. 2A and 2B, are contained.

The material of the elastic body (rubber plate) may be the same material as the acoustic lens 203, or an elastic body, of which acoustic impedance is midway between the acoustic impedances of the object and the acoustic lens 203, may be used. For example, a rubber material, such as silicon rubber, polyurethane and RTV may be used. When the rubber plate 205 is molded, oxide particles made of titanium oxide (particles size: not more than 0.5 μm) are dispersed at 0.1 to 0.5 wt %. The content is preferably not more than about 0.5 wt %. According to one example, for the rubber plate 205, titanium oxide particles (particle size: 0.21 μm) coated with aluminum oxide, are dispersed in the polyurethane at a 0.21 wt % concentration. On the surface of this rubber plate 105, opposite the surface that contacts the object, titanium oxide film is formed 100 Å as primer, and a gold film is formed thereon at a 1500 Å thickness, whereby the reflection film 204 is formed.

When the PAI is performed, the user installs the attachment 8 so that the surface of the reflection film 204 and the surface of the acoustic lens 203 contact with each other. Further, upon this installation, it is preferable that an acoustic matching agent (e.g. gel, water) is lightly coated between the attachment 8 and the acoustic lens 203.

By using the attachment 8, as illustrated in FIG. 4, the acoustic wave generated from the rubber plate 205 by the light emitted from the light source 3 decreases. Further, even if the illumination light enters inside the rubber plate 205, the illumination light is reflected by the reflection film 204, and does not enter the acoustic lens 203 and the acoustic matching layer 202. As a result, the photoacoustic wave, which may become a noise, can be decreased. This means that the signals that propagate inside the object, are reflected and scattered and are then received by the probe again, also decrease. As a consequence, the SNR of the photoacoustic signal improves. The contrast of the object information also improves.

According to Embodiment 2, a dedicated probe for PAI is unnecessary, and a standard probe for ultrasonic echo measurement available on the market can be used as the photoacoustic probe, hence cost can be reduced. A standard apparatus for ultrasonic echo measurement does not include a light source, so a light source for PAI should be provided separately. By installing the light source or an emitting end, to emit light from the light source, in the probe 201, or by controlling the light source or the light radiation from the emitting end, so that the unit operates interlocking with the probe, the probe for ultrasonic echo measurement can be used as the photoacoustic probe.

The shape of the attachment 8 preferably conforms to the surface shape of the acoustic lens 103, as illustrated in FIG. 4. For example, if the side of the probe 201 facing the object is a plane, it is preferable that the reflection film side of the attachment 8 is also a plane. However, if a rubber material of which modulus of elasticity is low, is used as a main component of the attachment 8, the attachment 8 may be manufactured as a plane shape, and be installed deforming along the object side surface when the attachment 8 is installed in the probe 201.

In FIG. 4, it is assumed that the elastic body disposed on the probe side (first elastic body) is the acoustic lens 203, but the first elastic body may be a flat elastic body, similarly to the rubber plate 105 in FIGS. 3A and 3B. In this case as well, the rubber plate 205 described in FIG. 4 can be used as the attachment side elastic body (second elastic body). In the case when the probe side elastic body (first elastic body) is the acoustic lens, the attachment side elastic body (second elastic body) may have a shape which does not lower the acoustic lens affect. For example, the first elastic body and the second elastic body may be integrated as one acoustic lens.

### Embodiment 3

In Embodiments 1 and 2, a diffusing body containing oxide particles, such as the acoustic lens 103 and the rubber plate 105 or 205, is used as the elastic body facing the object. However, the member that faces and contacts the object is not limited to the diffusing body. For example, a transparent body which transmits at least a part of the light having wavelengths emitted from the light source 3 may be used.

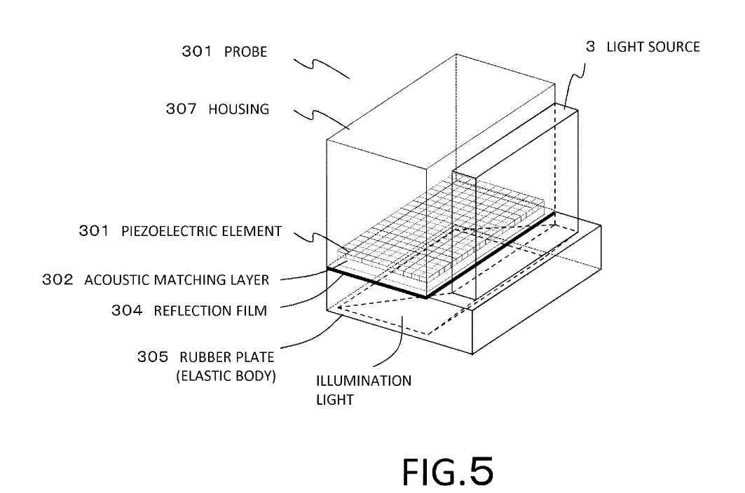

According to one example, when an illumination light having an 800 nm wavelength is used, a reflection film is formed on an elastic body (generated by mixing a hardening agent with silicon oil) on the surface at the opposite side of the surface contacting the object. Thereby the photoacoustic wave generated when the light contacts the probe 301 can be reduced. This mode is effective when, as shown in FIG. 5, the irradiation position of the illumination light in the housing 307 is directly below the piezoelectric element 101, since the illumination light can be efficiently guided to the object. In this case, the thickness of the rubber plate 105 is preferably at least ¼ the wavelength of the propagating ultrasonic wave, such as several mm to several ten mm.

If the transparent elastic body is used like this, the illumination light can be radiated to a region very close to the piezoelectric element 101, hence the reception intensity of the photoacoustic wave emitted from inside the object can be increased. As a result, the SNR can be improved.

As described above, according to the embodiments of the present invention, the photoacoustic waves, which are generated from the member of the probe facing the object and from the acoustic matching layer inside the probe, can be reduced. Therefore, noise generated because of these photoacoustic waves can be reduced, and the SNR of the photoacoustic acquisition signal and the contrast of the photoacoustic image improves.

According to the present invention, the SNR in the photoacoustic imaging can be improved.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-155556, filed on Aug. 10, 2017, which is hereby incorporated by reference herein in its entirety.

What is claimed is:

1. A photoacoustic probe, comprising:
a reception element configured to receive an acoustic wave generated in and propagated from an object which is irradiated with light;
an elastic body configured to contact with the object when receiving the acoustic wave and to diffuse or transmit the light having a wavelength that generates the acoustic wave;
an acoustic matching layer disposed between the elastic body and the reception element; and
a reflection film disposed between the acoustic matching layer and the elastic body.

2. The photoacoustic probe according to claim 1, wherein the elastic body is made of a resin material, and has a thickness that is at least approximately ¼ the wavelength of the propagating acoustic wave.

3. The photoacoustic probe according to claim 2, wherein the resin material is any of silicon rubber, polyurethane, and RTV.

4. The photoacoustic probe according to claim 1, wherein the elastic body is a diffusing body containing oxide particles, and a particle size of the oxide particle is not more than 0.5 μm, and the content of the oxide particles is not more than 0.5 wt %.

5. The photoacoustic probe according to claim 4, wherein the oxide particle is titanium oxide.

6. The photoacoustic probe according to claim 1, wherein the reflection film is a metal film or a dielectric film, and is disposed on the object side of the acoustic matching later or on the opposite side of the surface of the elastic body contacting the object.

7. The photoacoustic probe according to claim 6, wherein the reflection film is disposed on the acoustic matching layer or on the elastic body by film forming or adhesion.

8. The photoacoustic probe according to claim 1, wherein the elastic body is an acoustic lens or a rubber plate.

9. A photoacoustic probe comprising an ultrasonic probe for ultrasonic echo measurement, and an attachment to be combined with the ultrasonic probe, wherein
the ultrasonic probe includes:
a transmission/reception element configured to transmit an ultrasonic wave to an object and to receive the ultrasonic wave reflected by the object;
a first elastic body contacting the object when the ultrasonic wave is transmitted to the object; and
an acoustic matching layer disposed between the first elastic body and the transmission/reception element, and wherein
the attachment includes:
a second elastic body contacting the object when the ultrasonic probe and the attachment are combined, and diffusing or transmitting light having a wavelength that generates the acoustic wave; and
a reflection film disposed between the second elastic body and the first elastic body when the ultrasonic probe and the attachment are combined.
